(19) United States
(12) Patent Application Publication
(10) Pub. No.: US 2025/0302777 A1
(43) Pub. Date: Oct. 2, 2025

(54) METHODS FOR TREATING GASTROPARESIS

(71) Applicant: EVOKE PHARMA, INC., Solana Beach, CA (US)

(72) Inventors: Matthew J. D'ONOFRIO, Solana Beach, CA (US); David A. GONYER, Cardiff, CA (US)

(21) Appl. No.: 19/237,819

(22) Filed: Jun. 13, 2025

Related U.S. Application Data
(63) Continuation of application No. PCT/US2023/085127, filed on Dec. 20, 2023.
(60) Provisional application No. 63/476,356, filed on Dec. 20, 2022.

(51) Int. Cl. A61K 31/166

(52) U.S. Cl. CPC A61K 31/166

(57) ABSTRACT

Described herein are methods for treating gastroparesis with intranasally-administered compositions comprising metoclopramide. The methods include periods of time in which a patient is not administered a composition comprising metoclopramide.

### CROSS REFERENCE TO RELATED APPLICATIONS

This application is a continuation of International Application No. PCT/US2023/085127, filed Dec. 20, 2023, which claims benefit of priority to U.S. Provisional Application No. 63/476,356, filed Dec. 20, 2022, each of which is incorporated herein in its entirety.

### BACKGROUND OF THE INVENTION

Diabetic gastroparesis (DGP) is a chronic disorder of the stomach characterized by delayed gastric emptying and significant symptomatology of nausea, vomiting, early saticty, bloating, and/or severe abdominal pain. In 2020, the FDA approved the first metoclopramide (MCP) nasal outpatient treatment for patients with acute and recurrent DGP. However, there remains an unmet need for updated methods that reduce unwanted side effects. The present disclosure addresses this unmet need.

### SUMMARY OF THE INVENTION

The present disclosure provides methods for treating gastroparesis with intranasally-administered compositions comprising metoclopramide; the methods include periods of time in which a patient is not administered a composition comprising metoclopramide.

An aspect of the present disclosure is a method for treating chronic gastroparesis in a patient in need thereof. The method comprises steps of: intranasally administering to the patient a composition comprising metoclopramide, or a pharmaceutically-acceptable salt thereof, for a first period of time; after the first period of time, not administering to the patient a composition comprising metoclopramide, or a pharmaceutically-acceptable salt thereof, for a second period of time; and after the second period of time, intranasally administering to the patient a composition comprising metoclopramide, or a pharmaceutically-acceptable salt thereof, for a third period of time.

Another aspect of the present disclosure is a method for treating recurrent gastroparesis in a patient in need thereof. The method comprises steps of: intranasally administering to a patient a composition comprising metoclopramide, or a pharmaceutically-acceptable salt thereof. In this aspect, the patient was previously intranasally administered a composition comprising metoclopramide, or a pharmaceutically-acceptable salt thereof, for a first period of time, and the patient was not intranasally administered a composition comprising metoclopramide, or a pharmaceutically-acceptable salt thereof, for a second period of time after the first period of time.

A further aspect of the present disclosure is a method for reducing the likelihood or probability that a patient experiences tardive dyskinesia resulting from a metoclopramide therapy. The method comprises steps of: intranasally administering to the patient a composition comprising metoclopramide, or a pharmaceutically-acceptable salt thereof, for a first period of time; after the first period of time, not administering to the patient a composition comprising metoclopramide, or a pharmaceutically-acceptable salt thereof, for a second period of time; and after the second period of time, intranasally administering to the patient a composition comprising metoclopramide, or a pharmaceutically-acceptable salt thereof, for a third period of time.

An additional aspect of the present disclosure is a method for reducing the likelihood or probability that a patient experiences tardive dyskinesia resulting from a metoclopramide therapy. The method comprises steps of: intranasally administering to a patient a composition comprising metoclopramide, or a pharmaceutically-acceptable salt thereof. In this aspect, the patient was previously intranasally administered a composition comprising metoclopramide, or a pharmaceutically-acceptable salt thereof, for a first period of time, and the patient was not intranasally administered a composition comprising metoclopramide, or a pharmaceutically-acceptable salt thereof, for a second period of time after the first period of time.

In an aspect, the present disclosure provides a method for reducing the likelihood or probability that a patient experiences an adverse reaction resulting from a metoclopramide therapy. The method comprises steps of: intranasally administering to the patient a composition comprising metoclopramide, or a pharmaceutically-acceptable salt thereof, for a first period of time; after the first period of time, not administering to the patient a composition comprising metoclopramide, or a pharmaceutically-acceptable salt thereof, for a second period of time; and after the second period of time, intranasally administering to the patient a composition comprising metoclopramide, or a pharmaceutically-acceptable salt thereof, for a third period of time. In this aspect, the adverse reaction is one or more of an extrapyramidal effect, a neuroleptic malignant syndrome, depression, hypertension, fluid retention, hyperprolactionemia, and the inability to drive or operate machinery.

In another aspect, the present disclosure provides a method for reducing the likelihood or probability that a patient experiences an adverse reaction resulting from a metoclopramide therapy The method comprises steps of: intranasally administering to a patient a composition comprising metoclopramide, or a pharmaceutically-acceptable salt thereof. In this aspect, the patient was previously intranasally administered a composition comprising metoclopramide, or a pharmaceutically-acceptable salt thereof, for a first period of time. Also, the patient is not intranasally administered a composition comprising metoclopramide, or a pharmaceutically-acceptable salt thereof, for a second period of time after the first period of time; and the adverse reaction is one or more of an extrapyramidal effect, a neuroleptic malignant syndrome, depression, hypertension, fluid retention, hyperprolactionemia, and the inability to drive or operate machinery.

In embodiments, the method may comprise a third, a fourth, a fifth, a sixth, or a seventh period of time which are similar to the first period of time or the second period of time, as described above.

In embodiments, the patient is a human, e.g., a female human. In various embodiments, the patient, e.g., female patient, has diabetic gastroparesis.

In various embodiments, reducing the likelihood or probability is an at least 10% reduction in likelihood or probability, an least 20% reduction in likelihood or probability, an at least 30% reduction in likelihood or probability, an at least 40% reduction in likelihood or probability, an at least 50% reduction in likelihood or probability, an at least 60% reduction in likelihood or probability, an at least 70% reduction in likelihood or probability, an at least 80% reduction in likelihood or probability, or an at least 90% reduction in likelihood or probability. In some cases, reducing the likelihood or probability is an at least a one-fold reduction in likelihood or probability, at least a two-fold reduction in likelihood or probability, at least a three-fold reduction in likelihood or probability, at least a four-fold reduction in likelihood or probability, at least a five-fold reduction in likelihood or probability, at least a six-fold reduction in likelihood or probability, at least a seven-fold reduction in likelihood or probability, at least an eight-fold reduction in likelihood or probability, at least a nine-fold reduction in likelihood or probability, or at least a ten-fold reduction in likelihood or probability.

In embodiments, the patient does not experience tardive dyskinesia symptoms while the method is performed. In some embodiments, the patient does not experience adverse reaction symptoms while the method is performed; in these embodiments, the adverse reaction is one or more of an extrapyramidal effect, a neuroleptic malignant syndrome, depression, hypertension, fluid retention, hyperprolactionemia, and the inability to drive or operate machinery.

Additionally, any composition or method disclosed herein is applicable to any herein-disclosed composition or method. In other words, any aspect or embodiment described herein can be combined with any other aspect or embodiment as disclosed herein.

### DETAILED DESCRIPTION

The present disclosure provides methods for treating gastroparesis with intranasally-administered compositions comprising metoclopramide; the methods include periods of time in which a patient is not administered a composition comprising metoclopramide.

### Gastroparesis and Symptoms of Gastroparesis

Described herein are methods relating to treatment of gastroparesis and symptoms of gastroparesis. Gastroparesis can be described as a disorder that slows or stops the movement of food from the stomach to the small intestine. A subject may be suspected of having gastroparesis if the subject exhibits or has exhibited a symptom of gastroparesis. Some symptoms of gastroparesis are selected from the group consisting of: nausea (feeling sick to your stomach as if you were going to vomit or throw up); retching (heaving as if to vomit, but nothing comes up); vomiting; stomach fullness; not able to finish a normal-sized meal; feeling excessively full after meals; loss of appetite; bloating; stomach or belly visibly larger; and upper abdominal pain (above the navel); upper abdominal discomfort (above the navel). Some embodiments relate to a method of treating two, three, four, five, six, seven, eight, nine, ten, or eleven of the symptoms selected from the group consisting of: nausea (feeling sick to your stomach as if you were going to vomit or throw up); retching (heaving as if to vomit, but nothing comes up); vomiting; stomach fullness; not able to finish a normal-sized meal; feeling excessively full after meals; loss of appetite; bloating; stomach or belly visibly larger; upper abdominal pain (above the navel); and upper abdominal discomfort (above the navel). In some embodiments, the gastroparesis is diabetic gastroparesis.

In some embodiments, the patient in need thereof, has symptoms of nausea, bloating, emesis, delayed emesis, early satiety, vomiting, feeling full, loss of appetite, stomach fullness, stomach being visibly larger, and upper abdominal discomfort. In some cases, the symptoms are related to diabetic gastroparesis.

Methods of Treatment with Nasally Administered Metoclopramide

As used herein “metoclopramide” refers to metoclopramide in a solution formulation, including a salt of metoclopramide. In quantitating the mass of metoclopramide herein, unless otherwise specified, all masses of metoclopramide refer to the mass of the free base, which has a molecular weight of 299.80. One method of manufacturing metoclopramide is described in U.S. Pat. No. 3,177,252, which is incorporated herein by reference in its entirety. Thus, unless otherwise specified herein, the term “metoclopramide” includes the free base of metoclopramide (4-amino-5-chloro-N-[2-(diethylamino)ethyl]-2-methoxybenzamide) and pharmaceutically acceptable salts of metoclopramide free base. Where the “free base” or a specific salt of metoclopramide is intended, it is so specified. A particularly preferred form of metoclopramide is metoclopramide hydrochloride.

In embodiments, the composition comprising metoclopramide, or a pharmaceutically-acceptable salt thereof, further comprises benzalkonium chloride. In some cases, the composition has a concentration of benzalkonium chloride from about 0.005% (w/v) to about 0.05% (w/v). The benzalkonium chloride may be at a concentration of from about 0.02% to about 0.04% (w/v), e.g., from about 0.02% to about 0.03% (w/v). In some cases, the benzalkonium chloride is at a concentration of at least about 0.025% (w/v) or the benzalkonium chloride is at a concentration of about 0.025% (w/v).

In some embodiments, the composition comprising metoclopramide, or a pharmaceutically-acceptable salt thereof, further comprises benzyl alcohol. The benzyl alcohol may be at a concentration from about 0.01% (w/v) to about 1% (w/v). In some cases, the benzyl alcohol is at a concentration of about 0.75% (w/v).

In various embodiments, the composition comprising metoclopramide, or a pharmaceutically-acceptable salt thereof, further comprises a buffer. The buffer may be selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, IVIES (2-(N-morpholino)ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO (N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS (N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS (N-tris(hydroxy-methypmethyl-3-aminopropanesulfonic acid), and AMPD (2-amino-2-methyl-1,3-propanediol) buffer.

In several embodiments, the buffer comprises a citrate buffer. The citrate buffer may comprise a combination of citric acid monohydrate and sodium citrate dihydrate. In some cases, the citric acid monohydrate is in an amount of from about 0.2% to about 0.5% w/v, from about 0.25% to about 0.4% w/v, or from about 0.3% to about 0.35% w/v and the sodium citrate dihydrate is in an amount from about 1.0 to about 1.8% w/v, from about 1.2 to about 1.6% w/v, or from about 1.3 to about 1.5% w/v. A combined amount of citric acid monohydrate and sodium citrate dihydrate in the composition may be less than about 2.3% w/v. In various cases, the citric acid monohydrate is in an amount of about 0.1% and the sodium citrate dihydrate is in an amount of about 0.44%. In some cases, the composition provides a citrate concentration of at least about 10 millimolar.

In some cases, the buffer comprises sodium acetate.

In embodiments, the composition comprising metoclopramide, or a pharmaceutically-acceptable salt thereof, has a pH of above about 4.5, e.g., the composition has a pH of above about 4.6 or the composition has a pH of above about 5.0.

In some embodiments, the composition comprising metoclopramide, or a pharmaceutically-acceptable salt thereof, further comprises at least one member of the group consisting of a salt, edetate disodium dihydrate (EDTA), sorbitol, a sugar, and a flavoring agent.

In various embodiments, the composition comprising metoclopramide, or a pharmaceutically-acceptable salt thereof, is substantially free of an additional antioxidant.

In several embodiments, the composition comprising metoclopramide, or a pharmaceutically-acceptable salt thereof, has an osmolality of from about 500 mOsm/kg to about 1400 mOsm/kg.

In embodiments, the composition comprising metoclopramide, or a pharmaceutically-acceptable salt thereof, comprises benzalkonium chloride, citric acid monohydrate, edetate disodium dihydrate (EDTA), purified water, sodium citrate dihydrate, and sorbitol. In some cases, each 70 μL aliquot of the composition comprises 15 mg of metoclopramide, or a pharmaceutically-acceptable salt thereof; each 70 μL aliquot of the composition comprises 7.5 mg of metoclopramide, or a pharmaceutically-acceptable salt thereof; or each 35 μL aliquot of the composition comprises 7.5 mg of metoclopramide. In various cases, the composition has a pH of about 5.5. The composition may have a citrate concentration ([citrate]=[citric acid]+[dihydrogen citrate ion]+[hydrogen citrate ion]+[citrate ion]) of at least about 10 millimolar.

In some embodiments, the composition comprising metoclopramide, or a pharmaceutically-acceptable salt thereof, comprises benzyl alcohol, citric acid monohydrate, edetate disodium dihydrate (EDTA), purified water, sodium citrate dihydrate, and sorbitol. In some cases, the composition comprises less than about 1% w/v benzyl alcohol.

In various embodiments, the composition comprising metoclopramide, or a pharmaceutically-acceptable salt thereof, comprises benzyl alcohol, an acetate buffer, edetate disodium dihydrate (EDTA), purified water, and sorbitol.

In several embodiments, the composition comprising metoclopramide, or a pharmaceutically-acceptable salt thereof, exhibits less than about 2% average change in percent optical density (O.D.) per week per 200 mg/mL of metoclopramide when stored at a temperature of 40° C. and 75% relative humidity. In some cases, the average change of percent optical density (O.D.) is less than about 1.8% O.D. per week.

In embodiments, the composition comprising metoclopramide, or a pharmaceutically-acceptable salt thereof, is a nasal solution that remains clear to pale yellow when compared to standard E, 32 USP <631> on storage at a temperature of about 40° C. for at least about 8 weeks.

In some embodiments, the method comprises treatment of gastroparesis, including moderate to severe gastroparesis, of varying etiology, including gastroparesis arising out, associated with or caused by diabetes (including type 1 and type 2), postviral syndromes, anorexia nervosa, surgery on the stomach or vagus nerve, medications, such as anticholinergic and narcotic medications, which tend to suppress intestinal and gastroesophageal contractions, gastroesophageal reflux disease, smooth muscle disorders (e.g., amyloidosis and scleroderma), nervous system diseases (including abdominal migraine and Parkinson's disease), and/or metabolic disorders (including hypothyroidism). In some embodiments, the gastroparesis is moderate gastroparesis. In some embodiments, the gastroparesis is severe gastroparesis. In various embodiments, the patient is a human female, e.g., a human female with diabetes.

In some embodiments, nasal metoclopramide is administered in the absence of other gastroparesis medications. In some embodiments, additional medication may be administered if necessary. In some embodiments, the methods of treatment provided herein can also include co-administration of one or more additional therapeutic agents along with the metoclopramide nasal formulations described herein. The additional therapeutic agents administered concurrently with metoclopramide or at separate time intervals. In some embodiments, one or more other drugs may be incorporated into the metoclopramide nasal formulation. Additional therapeutic agents may include pain relievers, insulin and other drugs useful in the management of diabetes, steroids, especially steroids that prevent nasal irritation, and antidepressants.

Various techniques may be used to assess the severity of the gastroparesis and gastric emptying, and these will be well-known to those of skill in the art. Such techniques include questioning the patient regarding symptoms of gastroparesis by a Patient Reported Outcome (PRO) symptom measurement instrument. Techniques like octanoic breath test, wireless capsule endoscopy, radioscintigraphy, ultrasonography, and x-rays employing radiopaque markers such as barium, may be employed.

In embodiments, the composition comprising metoclopramide, or a pharmaceutically-acceptable salt thereof, is administered as at least two aliquots per day, e.g., the composition is administered as three aliquots per day, or the composition is administered as four aliquots per day.

In some embodiments, the composition comprising metoclopramide, or a pharmaceutically-acceptable salt thereof, is administered as an intranasal spray.

In several embodiments, an aliquot of the composition comprising metoclopramide, or a pharmaceutically-acceptable salt thereof, has a volume of from about 25 μL to about 140 μL, e.g., the aliquot of the composition has a volume of about 50 μL or the aliquot of the composition has a volume of about 70 μL.

In various embodiments, the composition comprising metoclopramide, or a pharmaceutically-acceptable salt thereof, is administered as one spray in one nostril or one spray in each nostril, from about 15 minutes to about 1 hour before a meal. The composition may be administered as one spray in one nostril, from about 20 minutes to about 45 minutes before a meal. In some cases, the composition is administered as one spray in one or each nostril, about 30 minutes before a meal.

Administration may be prescribed 30 minutes before meals, assuming three meals per day, and before bedtime. In some embodiments, doses are administered before breakfast and dinner. In some embodiments, each dose is administered as a single intranasal aliquot (e.g., spray); in some embodiments, each dose is administered as two aliquots (e.g., one spray per nostril). The nasal metoclopramide compositions described herein may be administered to a patient, e.g., a human female patient, as one spray in a single nostril, four times a day (one spray QID for about 1, 2, 3, 4, 5, 6, 7, or 8 weeks), or one spray per nostril in both nostrils four times a day (two sprays QID for about 1, 2, 3, 4, 5, 6, 7, or 8 weeks).

In several embodiments, the composition has a concentration of metoclopramide, or a pharmaceutically-acceptable salt thereof, of from about 20.0% (w/v) to about 30.0% (w/v). The composition may comprise 5 mg to 25 mg of metoclopramide, or a pharmaceutically-acceptable salt thereof, per aliquot. In some cases, In some cases, the metoclopramide composition comprises about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, of metoclopramide, or a pharmaceutically-acceptable salt thereof, per aliquot. A dose of 20 mg to 100 mg of metoclopramide, or a pharmaceutically-acceptable salt thereof, may be administered per day, a dose of 30 mg to 80 mg of metoclopramide, or a pharmaceutically-acceptable salt thereof, may be administered per day, a dose of 30 mg to 60 mg of metoclopramide, or a pharmaceutically-acceptable salt thereof, may be administered per day, or a dose of 30 mg to 45 mg of metoclopramide, or a pharmaceutically-acceptable salt thereof, may be administered per day.

For example, a dose of between about 0.1 mg/kg to about 2.5 mg/kg may be administered to a patient having gastroparesis. Exemplary dosages can be about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg. In some embodiments, a nasal dosage is between about 0.06 to about 1.2 mg/kg of body weight. In some embodiments, the nasal dosages are about 0.06 mg/kg, 0.08 mg/kg, 1.0 mg/kg, 1.2 mg/kg and 1.4 mg/kg.

As the weight of the patient may affect the dosage to be administered, the person skilled in the art will know to vary or titrate the dose in order to obtain an optimal effect in relation to the dose tolerated by the patient.

In some embodiments, a clinician will prescribe a lower dosage of metoclopramide because of an underlying medical condition or other clinical consideration. For example, in the case of renal impairment, the clinician will prescribe a dose that is appropriate for the degree of renal impairment or other rationale for slower metabolism or clearance of the metoclopramide, e.g., a dose that is 25% to 75% lower, in some embodiments 50% lower, than the dose prescribed for a patient without renal impairment. In some such embodiments, the daily dose will be 20 mg administered as two intranasal doses, e.g., one dose before breakfast and one before dinner. In some embodiments, each dose is administered as a single intranasal aliquot (e.g., spray). In some embodiments, each dose is administered as two intranasal aliquots (e.g., two sprays, one in each nostril).

The aforementioned dosages for the treatment and control of gastroparesis may be administered before meals and/or before bedtime. In some embodiments, each dose is administered as a single intranasal aliquot (e.g., one spray in one nostril); in some embodiments, the dose may be split into two or more intranasal aliquots (e.g., two sprays, one in each nostril).

Methods for Avoiding or Reducing the Likelihood of Experiencing Side Effects Associated with Metoclopramide

The present disclosure provides methods for treating gastroparesis with intranasally-administered compositions comprising metoclopramide; the methods include periods of time in which a patient is not administered a composition comprising metoclopramida.

An aspect of the present disclosure is a method for treating chronic gastroparesis in a patient in need thereof. The method comprises steps of: intranasally administering to the patient a composition comprising metoclopramide, or a pharmaceutically-acceptable salt thereof, for a first period of time; after the first period of time, not administering to the patient a composition comprising metoclopramide, or a pharmaceutically-acceptable salt thereof, for a second period of time; and after the second period of time, intranasally administering to the patient a composition comprising metoclopramide, or a pharmaceutically-acceptable salt thereof, for a third period of time.

Another aspect of the present disclosure is a method for treating recurrent gastroparesis in a patient in need thereof. The method comprises steps of: intranasally administering to a patient a composition comprising metoclopramide, or a pharmaceutically-acceptable salt thereof. In this aspect, the patient was previously intranasally administered a composition comprising metoclopramide, or a pharmaceutically-acceptable salt thereof, for a first period of time, and the patient was not intranasally administered a composition comprising metoclopramide, or a pharmaceutically-acceptable salt thereof, for a second period of time after the first period of time.

A further aspect of the present disclosure is a method for reducing the likelihood or probability that a patient experiences tardive dyskinesia resulting from a metoclopramide therapy. The method comprises steps of: intranasally administering to the patient a composition comprising metoclopramide, or a pharmaceutically-acceptable salt thereof, for a first period of time; after the first period of time, not administering to the patient a composition comprising metoclopramide, or a pharmaceutically-acceptable salt thereof, for a second period of time; and after the second period of time, intranasally administering to the patient a composition comprising metoclopramide, or a pharmaceutically-acceptable salt thereof, for a third period of time.

An additional aspect of the present disclosure is a method for reducing the likelihood or probability that a patient experiences tardive dyskinesia resulting from a metoclopramide therapy. The method comprises steps of: intranasally administering to a patient a composition comprising metoclopramide, or a pharmaceutically-acceptable salt thereof. In this aspect, the patient was previously intranasally administered a composition comprising metoclopramide, or a pharmaceutically-acceptable salt thereof, for a first period of time, and the patient was not intranasally administered a composition comprising metoclopramide, or a pharmaceutically-acceptable salt thereof, for a second period of time after the first period of time.

In an aspect, the present disclosure provides a method for reducing the likelihood or probability that a patient experiences an adverse reaction resulting from a metoclopramide therapy. The method comprises steps of: intranasally administering to the patient a composition comprising metoclopramide, or a pharmaceutically-acceptable salt thereof, for a first period of time; after the first period of time, not administering to the patient a composition comprising metoclopramide, or a pharmaceutically-acceptable salt thereof, for a second period of time; and after the second period of time, intranasally administering to the patient a composition comprising metoclopramide, or a pharmaceutically-acceptable salt thereof, for a third period of time. In this aspect, the adverse reaction is one or more of an extrapyramidal effect, a neuroleptic malignant syndrome, depression, hypertension, fluid retention, hyperprolactionemia, and the inability to drive or operate machinery.

In another aspect, the present disclosure provides a method for reducing the likelihood or probability that a patient experiences an adverse reaction resulting from a metoclopramide therapy The method comprises steps of: intranasally administering to a patient a composition comprising metoclopramide, or a pharmaceutically-acceptable salt thereof. In this aspect, the patient was previously intranasally administered a composition comprising metoclopramide, or a pharmaceutically-acceptable salt thereof, for a first period of time. Also, the patient is not intranasally administered a composition comprising metoclopramide, or a pharmaceutically-acceptable salt thereof, for a second period of time after the first period of time; and the adverse reaction is one or more of an extrapyramidal effect, a neuroleptic malignant syndrome, depression, hypertension, fluid retention, hyperprolactionemia, and the inability to drive or operate machinery.

In embodiments, the method may comprise a third, a fourth, a fifth, a sixth, or a seventh period of time which are similar to the first period of time or the second period of time, as described above.

In embodiments, the sum of the first period of time and the third period of time is longer than 12 weeks.

In some embodiments, the first period of time is longer than 12 weeks and/or the third period of time is longer than 12 weeks.

In various embodiments, the first period of time of time is from about 2 weeks to about 8 weeks and/or the third period of time of time is from about 2 weeks to about 8 weeks.

In several embodiments, the second period of time is at least 5 days, is at least 6 days, or is at least 7 days, is at least 8 days, is at least 9 days, is at least 10 days, is at least 12 days, is at least 13 days, is at least 2 weeks, is at least 3 weeks, is at least 4 weeks, is at least two months, is at least three months, is at least four months, is at least five months, is at least six months, is at least seven months, is at least eight months, is at least nine months, is at least ten months, is at least eleven months, or is at least a year.

In embodiments, the method further comprising a step of: after the third period of time, not administering to the patient a composition comprising metoclopramide, or a pharmaceutically-acceptable salt thereof, for a fourth period of time. The fourth period of time may be at least 5 days, is at least 6 days, or is at least 7 days, is at least 8 days, is at least 9 days, is at least 10 days, is at least 12 days, is at least 13 days, is at least 2 weeks, is at least 3 weeks, is at least 4 weeks, is at least two months, is at least three months, is at least four months, is at least five months, is at least six months, is at least seven months, is at least eight months, is at least nine months, is at least ten months, is at least eleven months, or is at least a year.

In some cases, the method further comprising a step of: after the fourth period of time, intranasally administering to the patient a composition comprising metoclopramide, or a pharmaceutically-acceptable salt thereof, for a fifth period of time. The fourth period of time may be at least 5 days, is at least 6 days, or is at least 7 days, is at least 8 days, is at least 9 days, is at least 10 days, is at least 12 days, is at least 13 days, is at least 2 weeks, is at least 3 weeks, is at least 4 weeks, is at least two months, is at least three months, is at least four months, is at least five months, is at least six months, is at least seven months, is at least eight months, is at least nine months, is at least ten months, is at least eleven months, or is at least a year. The fifth period of time may be longer than 12 weeks or the fifth period of time may be from about 2 weeks to about 8 weeks.

In various cases, the method further comprising a step of after the fifth period of time, not administering to the patient a composition comprising metoclopramide, or a pharmaceutically-acceptable salt thereof, for a sixth period of time. The fifth period of time may be longer than 12 weeks or the fifth period of time may be from about 2 weeks to about 8 weeks. The sixth period of time may be at least 5 days, is at least 6 days, or is at least 7 days, is at least 8 days, is at least 9 days, is at least 10 days, is at least 12 days, is at least 13 days, is at least 2 weeks, is at least 3 weeks, is at least 4 weeks, is at least two months, is at least three months, is at least four months, is at least five months, is at least six months, is at least seven months, is at least eight months, is at least nine months, is at least ten months, is at least eleven months, or is at least a year.

In several cases, the method further comprising a step of after the sixth period of time, intranasally administering to the patient a composition comprising metoclopramide, or a pharmaceutically-acceptable salt thereof, for a seventh period of time. The sixth period of time may be at least 5 days, is at least 6 days, or is at least 7 days, is at least 8 days, is at least 9 days, is at least 10 days, is at least 12 days, is at least 13 days, is at least 2 weeks, is at least 3 weeks, is at least 4 weeks, is at least two months, is at least three months, is at least four months, is at least five months, is at least six months, is at least seven months, is at least eight months, is at least nine months, is at least ten months, is at least eleven months, or is at least a year. The seventh period of time may be longer than 12 weeks or the seventh period of time may be from about 2 weeks to about 8 weeks.

In some embodiments, the method is performed for greater than 12 weeks. In some cases, the method is performed for six months or longer, the method is performed for one year or longer, the method is performed for two years or longer, the method is performed for three years or longer, the method is performed for four years or longer, or the method is performed for five years or longer.

In various embodiments, reducing the likelihood or probability is an at least 10% reduction in likelihood or probability relative to a method lacking, at least, a second period of time.

In several embodiments, reducing the likelihood or probability is an at least 10% reduction in likelihood or probability relative to a method lacking, at least, a fourth period of time.

In embodiments, reducing the likelihood or probability is an at least 10% reduction in likelihood or probability relative to a method lacking, at least, a sixth period of time.

In embodiments, the composition comprising metoclopramide, or a pharmaceutically-acceptable salt thereof, further comprises benzalkonium chloride. In some cases, the composition has a concentration of benzalkonium chloride from about 0.005% (w/v) to about 0.05% (w/v). The benzalkonium chloride may be at a concentration of from about 0.02% to about 0.04% (w/v), e.g., from about 0.02% to about 0.03% (w/v). In some cases, the benzalkonium chloride is at a concentration of at least about 0.025% (w/v) or the benzalkonium chloride is at a concentration of about 0.025% (w/v).

In some embodiments, the composition comprising metoclopramide, or a pharmaceutically-acceptable salt thereof, further comprises benzyl alcohol. The benzyl alcohol may be at a concentration from about 0.01% (w/v) to about 1% (w/v). In some cases, the benzyl alcohol is at a concentration of about 0.75% (w/v).

In various embodiments, the composition comprising metoclopramide, or a pharmaceutically-acceptable salt thereof, further comprises a buffer. The buffer may be selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, IVIES (2-(N-morpholino)ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO (N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS (N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS (N-tris(hydroxy-methypmethyl-3-aminopropanesulfonic acid), and AMPD (2-amino-2-methyl-1,3-propanediol) buffer.

In several embodiments, the buffer comprises a citrate buffer. The citrate buffer may comprise a combination of citric acid monohydrate and sodium citrate dihydrate. In some cases, the citric acid monohydrate is in an amount of from about 0.2% to about 0.5% w/v, from about 0.25% to about 0.4% w/v, or from about 0.3% to about 0.35% w/v and the sodium citrate dihydrate is in an amount from about 1.0 to about 1.8% w/v, from about 1.2 to about 1.6% w/v, or from about 1.3 to about 1.5% w/v. A combined amount of citric acid monohydrate and sodium citrate dihydrate in the composition may be less than about 2.3% w/v. In various cases, the citric acid monohydrate is in an amount of about 0.1% and the sodium citrate dihydrate is in an amount of about 0.44%. In some cases, the composition provides a citrate concentration of at least about 10 millimolar.

In some cases, the buffer comprises sodium acetate.

In embodiments, the composition comprising metoclopramide, or a pharmaceutically-acceptable salt thereof, has a pH of above about 4.5, e.g., the composition has a pH of above about 4.6 or the composition has a pH of above about 5.0.

In some embodiments, the composition comprising metoclopramide, or a pharmaceutically-acceptable salt thereof, further comprises at least one member of the group consisting of a salt, edetate disodium dihydrate (EDTA), sorbitol, a sugar, and a flavoring agent.

In various embodiments, the composition comprising metoclopramide, or a pharmaceutically-acceptable salt thereof, is substantially free of an additional antioxidant.

In several embodiments, the composition has a concentration of metoclopramide, or a pharmaceutically-acceptable salt thereof, of from about 20.0% (w/v) to about 30.0% (w/v). The composition may comprise 5 mg to 25 mg of metoclopramide, or a pharmaceutically-acceptable salt thereof, per aliquot. In some cases, In some cases, the metoclopramide composition comprises about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, of metoclopramide, or a pharmaceutically-acceptable salt thereof, per aliquot. A dose of 20 mg to 100 mg of metoclopramide, or a pharmaceutically-acceptable salt thereof, may be administered per day, a dose of 30 mg to 80 mg of metoclopramide, or a pharmaceutically-acceptable salt thereof, may be administered per day, a dose of 30 mg to 60 mg of metoclopramide, or a pharmaceutically-acceptable salt thereof, may be administered per day, or a dose of 30 mg to 45 mg of metoclopramide, or a pharmaceutically-acceptable salt thereof, may be administered per day.

In embodiments, the composition comprising metoclopramide, or a pharmaceutically-acceptable salt thereof, is administered as at least two aliquots per day, e.g., the composition is administered as three aliquots per day, or the composition is administered as four aliquots per day.

In some embodiments, the composition comprising metoclopramide, or a pharmaceutically-acceptable salt thereof, is administered as an intranasal spray.

In several embodiments, an aliquot of the composition comprising metoclopramide, or a pharmaceutically-acceptable salt thereof, has a volume of from about 25 μL to about 140 μL, e.g., the aliquot of the composition has a volume of about 50 μL or the aliquot of the composition has a volume of about 70 μL.

In various embodiments, the composition comprising metoclopramide, or a pharmaceutically-acceptable salt thereof, is administered as one spray in one nostril or one spray in each nostril, from about 15 minutes to about 1 hour before a meal. The composition may be administered as one spray in one nostril, from about 20 minutes to about 45 minutes before a meal. In some cases, the composition is administered as one spray in one or each nostril, about 30 minutes before a meal.

In several embodiments, the composition comprising metoclopramide, or a pharmaceutically-acceptable salt thereof, has an osmolality of from about 500 mOsm/kg to about 1400 mOsm/kg.

In embodiments, the composition comprising metoclopramide, or a pharmaceutically-acceptable salt thereof, comprises benzalkonium chloride, citric acid monohydrate, edetate disodium dihydrate (EDTA), purified water, sodium citrate dihydrate, and sorbitol. In some cases, each 70 μL aliquot of the composition comprises 15 mg of metoclopramide, or a pharmaceutically-acceptable salt thereof; each 70 μL aliquot of the composition comprises 7.5 mg of metoclopramide, or a pharmaceutically-acceptable salt thereof; or each 35 μL aliquot of the composition comprises 7.5 mg of metoclopramide. In various cases, the composition has a pH of about 5.5. The composition may have a citrate concentration ([citrate]=[citric acid]+[dihydrogen citrate ion]+[hydrogen citrate ion]+[citrate ion]) of at least about 10 millimolar.

In some embodiments, the composition comprising metoclopramide, or a pharmaceutically-acceptable salt thereof, comprises benzyl alcohol, citric acid monohydrate, edetate disodium dihydrate (EDTA), purified water, sodium citrate dihydrate, and sorbitol. In some cases, the composition comprises less than about 1% w/v benzyl alcohol.

In various embodiments, the composition comprising metoclopramide, or a pharmaceutically-acceptable salt thereof, comprises benzyl alcohol, an acetate buffer, edetate disodium dihydrate (EDTA), purified water, and sorbitol.

In several embodiments, the composition comprising metoclopramide, or a pharmaceutically-acceptable salt thereof, exhibits less than about 2% average change in percent optical density (O.D.) per week per 200 mg/mL of metoclopramide when stored at a temperature of 40° C. and 75% relative humidity. In some cases, the average change of percent optical density (O.D.) is less than about 1.8% O.D. per week.

In embodiments, the composition comprising metoclopramide, or a pharmaceutically-acceptable salt thereof, is a nasal solution that remains clear to pale yellow when compared to standard E, 32 USP <631> on storage at a temperature of about 40° C. for at least about 8 weeks.

In some embodiments, the patient does not experience tardive dyskinesia symptoms while the method is performed.

In various embodiments, the patient does not experience adverse reaction symptoms while the method is performed. In this embodiment, the adverse reaction is one or more of an extrapyramidal effect, a neuroleptic malignant syndrome, depression, hypertension, fluid retention, hyperprolactionemia, and the inability to drive or operate machinery.

In embodiments, the patient is a human, e.g., a female human. In various embodiments, the patient, e.g., female patient, has diabetic gastroparesis.

In embodiments, the patient in need thereof, has symptoms of nausea, bloating, emesis, delayed emesis, early satiety, vomiting, feeling full, loss of appetite, stomach fullness, stomach being visibly larger, and upper abdominal discomfort. In some cases, the symptoms are related to diabetic gastroparesis.

In several embodiments, reducing the likelihood or probability is an at least 10% reduction in likelihood or probability, an least 20% reduction in likelihood or probability, an at least 30% reduction in likelihood or probability, an at least 40% reduction in likelihood or probability, an at least 50% reduction in likelihood or probability, an at least 60% reduction in likelihood or probability, an at least 70% reduction in likelihood or probability, an at least 80% reduction in likelihood or probability, or an at least 90% reduction in likelihood or probability. In some cases, reducing the likelihood or probability is an at least a one-fold reduction in likelihood or probability, at least a two-fold reduction in likelihood or probability, at least a three-fold reduction in likelihood or probability, at least a four-fold reduction in likelihood or probability, at least a five-fold reduction in likelihood or probability, at least a six-fold reduction in likelihood or probability, at least a seven-fold reduction in likelihood or probability, at least an eight-fold reduction in likelihood or probability, at least a nine-fold reduction in likelihood or probability, or at least a ten-fold reduction in likelihood or probability.

### Manufacture of Nasal Formulations

Nasal compositions of metoclopramide may be manufactured for administration as a medicament for administration to a patient for one of the indications described herein. In some embodiments, the nasal metoclopramide formulation is one described in U.S. Pat. No. 8,334,281, which is incorporated herein in its entirety. Briefly, metoclopramide, buffer, benzalkonium chloride and optionally other ingredients (such as sodium chloride or other osmolarity-regulating agent, sorbitol or other sweetener, flavoring agent, etc.) may be made up to some volume less than the target final volume of the solution. The ingredients may then be mixed until all the ingredients are dissolved. The pH then may be adjusted, if necessary, by addition of a suitable acid or base, such as HCl, NaOH, or the complementary acid or base of the buffer. Once the desired pH has been obtained, the solution may then be brought up to full volume with water. The resulting solution may then be packaged in a suitable container for shipping and distribution. In some embodiments, the suitable container includes a nasal pump as described in more detail below. In other embodiments, the suitable container may be a vial, such as an amber glass vial, which may be a glass ampule, a glass bottle topped with an inert rubber septum and crimp cap top, or another suitable pharmaceutical vial.

To combat the deleterious effects of light on metoclopramide, the manufacture may conveniently include a container, especially an opaque container, i.e., a container that is at least partially or completely impervious to light. In some embodiments, a suitable opaque container will be brown or amber, especially brown or amber glass. In other embodiments, the opaque container will be an opaque polymer container, such as is commonly used in the pharmaceutical arts.

Some embodiments described herein provide, as a manufacture, a combination of a stable, clear and/or colorless solution of metoclopramide and a means for intranasal administration of the metoclopramide solution. In some embodiments, the manufacture comprises one of the metoclopramide solutions described herein and an intranasal delivery device comprising a reservoir, in which the metoclopramide solution is contained, a pump in fluid communication with the reservoir and a nozzle in fluid communication with the pump. In use, the pump is actuated, drawing an amount of the metoclopramide solution from the reservoir and expelling the solution out of the nozzle as an aerosolized spray. Suitable nasal administration devices are commercially available. Among the suppliers of nasal administration devices that may be combined with a stable, substantially clear and/or substantially colorless metoclopramide solution according to the present invention, there may be mentioned Aptar (Valois of America, Congers, New York, and Pfeiffer of America, Princeton, N.J.) In some embodiments, the intranasal delivery device is partially or completely opaque, in order to protect the contents of the device from exposure to ambient light.

As used herein, a “nasal administration device” is a device capable of administering a dose of a composition comprising metoclopramide into the nose of a patient. In some embodiments, the nasal administration device is an atomizer, comprising a reservoir adapted to contain the metoclopramide solution and a pump adapted to draw a predetermined amount of the metoclopramide solution from the reservoir dispense the predetermined amount of metoclopramide solution through an atomizing nozzle and into at least one nostril of a patient. Suitable nasal administration devices are commercially available.

As used herein, the term “spray” indicates an atomized volume of liquid expelled from a nozzle of a nasal administration device upon a single activation of the nasal administration device. In general, each spray is administered into a single nostril of a patient. As such, a “spray”, as used herein, is a type of “aliquot”, the latter being a generic term referring to an amount of liquid sprayed, instilled, or otherwise introduced into a nostril of a subject, such as a patient.

### Stability of Nasal Compositions of Metoclopramide

Compositions described herein comprise metoclopramide, or a pharmaceutically acceptable salt thereof, in a stable composition. In some embodiments described herein, stable metoclopramide solutions are solutions containing metoclopramide characterized by color stability or clarity of the solution. In some embodiments, color stability refers to the tendency of a formulated solution to maintain the same color, or absence of color, upon storage for a predetermined period of time as it had when originally formulated. In some embodiments, stability refers to the tendency of a formulated solution to maintain the same clarity upon storage for a predetermined period of time as it had when originally formulated. In some embodiments, stability refers to the tendency of a formulated solution to resist degradation of one or more ingredients, and in particular metoclopramide, during storage. In some embodiments, such compositions are stable for a period of at least about 1 month, at least about 2 months, at least about 3 months, at least about 4 months, at least about 5 months, at least about 6 months, at least about 9 months, at least about 12 months, at least about 15 months, at least about 18 months, at least about 21 months or at least about 24 months at temperatures in the range of about 5° C. to about 25° C. In some embodiments, long-term storage at 5° C. to 25° C. may be simulated under accelerated conditions, e.g., at a temperature in the range of about 35° C. to about 60° C., particularly in a range of about 35° C. to about 45° C., e.g., about 40° C. Thus, in some embodiments, the nasal compositions of metoclopramide provided herein are stable upon storage under accelerated conditions, e.g., at about 25° C. to about 60°, especially at about 30° C. to about 50° C., about 35° C. to about 45° C. or about 40° C. for at least about 1 week, at least about 2 weeks, at least about 3 weeks, at least about 4 weeks, at least about 5 weeks, at least about 6 weeks, at least about 9 weeks, at least about 12 weeks, at least about 15 weeks, at least about 18 weeks, at least about 21 weeks or at least about 24 weeks.

Stability may be determined by methods known in the art, such as those dictated by the United States Pharmacopoeia (USP). In particular, USP 26, pages 500-502, and 2138-2140 (incorporated herein by reference) provide general procedures for preparing standard colored solutions for color determination and for determining the color or achromicity of a solution. Thus, the person skilled in the art will know how to prepare standard solutions and compare the color of a composition of the invention against standard solutions. 32 USP <631>, pages 238-239 (incorporated herein by reference) provide standardized methods for measuring the stability of metoclopramide in injectable and oral solutions of metoclopramide. The person skilled in the art would thus know how to test the stability of metoclopramide compositions. Color standards can also include standards A, B, C, D and/or E described in 32 USP <631>. In some embodiments, another color standard that may be useful for determining the stability of nasal metoclopramide solutions is a 50:50 dilution of standard C with distilled water, wherein C is as described in 32 USP <631>. The 50:50 dilution of C with distilled water is also referred to herein at 0.5 C, and can be prepared by combining 1 mL Cobaltous Chloride CS, 6 mL Ferric Chloride CS, 1 mL Cupric Sulfate CS, and q.s. to 50 mL with distilled water to produce standard C and then diluting C to 0.5 C by combining 1 part C with 1 part distilled water. A solution that is lighter than 0.5 C is considered to be substantially clear as the term is used herein. The Cobaltous Chloride CS, Ferric Chloride CS and Cupric Sulfate CS are colorometric solutions are commercially available; they may also be prepared according to 32 USP under Colorimetric Solutions (CS) in the section Reagents, Indicators, and Solutions, which is incorporated by reference herein in its entirety. In some other preferred embodiments, the color reference standard is “E” from 32 USP <631>. The standard matching solution “E” is prepared by combining 4.0 mL of cobaltous chloride colorimetric solution (USP CS), 12.0 mL of ferric chloride colorimetric solution (USP CS), and 3.0 mL of cupric sulfate solution (USP CS) into a 50 mL volumetric flask and making the flask up to 50 mL with deionized water. Color determination is conducted by pipetting 5.0 mL of standard matching solution into a 20 mL scintillation vial (about 15 mm height), pipetting 5.0 mL of sample solution into a separate 20 mL scintillation vial (about 15 mm height) and comparing the color of the two solutions under diffused day light against a vertical white background. In some embodiments, a sample whose color is clear, lighter than the standard or the same color as the standard is considered “substantially free of color”, “substantially clear” or “stable” as described herein. Objectivity may be ensured by having the color of a test solution evaluated against the standard solution by more than one person.

In some embodiments, the metoclopramide compositions described herein are colorless when formulated and remain colorless for a period of at least about 2 weeks, about 1 month, at least about 2 months, at least about 3 months, at least about 4 months, at least about 5 months, at least about 6 months, at least about 9 months, at least about 12 months, at least about 15 months, at least about 18 months, at least about 21 months or at least about 24 months at temperatures in the range of about 5° C. to about 25° C. In some embodiments, long-term storage at 5° C. to 25° C. may be simulated under accelerated conditions, e.g., at a temperature in the range of about 35° C. to about 60° C., particularly in a range of about 35° C. to about 45° C., e.g., about 40° C. Thus, in some embodiments, the nasal compositions of metoclopramide provided herein are colorless when formulated and remain colorless upon storage under accelerated conditions, e.g., at about 25° C. to about 60°, especially at about 30° C. to about 50° C., about 35° C. to about 45° C. or about 40° C. for at least about 1 week, at least about 2 weeks, at least about 3 weeks, at least about 4 weeks, at least about 5 weeks, at least about 6 weeks, at least about 9 weeks, at least about 12 weeks, at least about 15 weeks, at least about 18 weeks, at least about 21 weeks or at least about 24 weeks.

In several embodiments, the composition comprising metoclopramide, or a pharmaceutically-acceptable salt thereof, exhibits less than about 2% average change in percent optical density (O.D.) per week per 200 mg/mL of metoclopramide when stored at a temperature of 40° C. and 75% relative humidity. In some cases, the average change of percent optical density (O.D.) is less than about 1.8% O.D. per week.

In embodiments, the composition comprising metoclopramide, or a pharmaceutically-acceptable salt thereof, is a nasal solution that remains clear to pale yellow when compared to standard E, 32 USP <631> on storage at a temperature of about 40° C. for at least about 8 weeks.

### Buffers

In various embodiments, the composition comprising metoclopramide, or a pharmaceutically-acceptable salt thereof, further comprises a buffer. The buffer may be selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, IVIES (2-(N-morpholino)ethanesulfonic acid), BIS-(bis(2-hydroxyethyl)iminotris(hydroxymethyl)methane), ADA (N-(2-acetamido)-2-TRIS iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO (N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS (N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS (N-tris(hydroxy-methypmethyl-3-aminopropanesulfonic acid), and AMPD (2-amino-2-methyl-1,3-propanediol) buffer. In some embodiments, other pharmaceutically acceptable buffers may be used.

In embodiments, the composition comprising metoclopramide, or a pharmaceutically-acceptable salt thereof, has a pH of above about 4.5, e.g., the composition has a pH of above about 4.6 or the composition has a pH of above about 5.0.

In several embodiments, the buffer comprises a citrate buffer. The citrate buffer may comprise a combination of citric acid monohydrate and sodium citrate dihydrate. In some cases, the citric acid monohydrate is in an amount of from about 0.2% to about 0.5% w/v, from about 0.25% to about 0.4% w/v, or from about 0.3% to about 0.35% w/v and the sodium citrate dihydrate is in an amount from about 1.0 to about 1.8% w/v, from about 1.2 to about 1.6% w/v, or from about 1.3 to about 1.5% w/v. A combined amount of citric acid monohydrate and sodium citrate dihydrate in the composition may be less than about 2.3% w/v. In various cases, the citric acid monohydrate is in an amount of about 0.1% and the sodium citrate dihydrate is in an amount of about 0.44%. In some cases, the composition provides a citrate concentration of at least about 10 millimolar.

In some cases, the buffer comprises sodium acetate.

In some embodiments, the nasal formulations of metoclopramide must have a pH of at least about 4.5, at least about 4.6, at least about 4.7, at least about 4.8, at least about 4.9, at least about 5, at least about 5.1 or at least about 5.2. In order to achieve and maintain an appropriate pH, it is considered necessary to use a buffer. Generally, a buffer comprises a combination of an acid (sometimes abbreviated HA) and a complementary base (A-). A buffer may be referred to by reciting the acid or the base that forms one half of the complementary acid-base (HA-A) pair. For example, acetic acid has the formula: CH₃C(O)OH, and forms a buffer with its complementary base, acetate ion, CH₃C(O)O— in aqueous solution. The buffer so formulated may be referred to as an acetate buffer or as an acetic acid buffer. The person having skill in the art will recognize that when the term buffer is used with either the acid or its complementary base, it refers to a mixture of the acid and the free base in solution. Thus, a citrate buffer (or citric acid buffer) refers to a mixture of citric acid and citrate ion. Since citric acid has three titratable groups, a citrate buffer (or citric acid buffer) can refer to a mixture of citric acid, and one or more of the complementary bases resulting from removal of one, two or three of the protons.

In some embodiments described herein, it is desirable to combine citric acid monohydrate and sodium citrate dihydrate in the nasal preparation in proportions suitable to provide a stable pH (+/−0.2 pH units) of about 4.5 and above, about 4.6 or above, about 4.7 or above, about 4.8 or above, about 4.9 or above, about 5 or above, about 5.1 or above, about 5.2 or above, about 5.3 or above, about 4.5 to about 6.0, about 4.6 to about 5.8, about 4.7 to about 5.6, about 4.5 to about 5.5, about 4.6 to about 5.7, about 4.7 to about 5.8, about 5 to about 5.7, about 5.1 to about 5.6. It is to be understood that the pH of the solution may vary slightly upon storage, e.g., at accelerated or ambient conditions. Variance of ±0.05 to ±0.4 pH units, ±0.1 to ±0.3 pH units or ±0.05 to ±0.25 pH units may be noted upon storage.

In some embodiments provided herein, it is desirable to combine glacial acetic acid and sodium acetate trihydrate in the nasal preparation in proportions suitable to provide a stable pH (+/−0.2 pH units) of about 4.5 and above, about 4.6 or above, about 4.7 or above, about 4.8 or above, about 4.9 or above, about 5 or above, about 5.1 or above, about 5.2 or above, about 5.3 or above, about 4.5 to about 6.0, about 4.6 to about 5.8, about 4.7 to about 5.6, about 4.5 to about 5.5, about 4.6 to about 5.7, about 4.7 to about 5.8, about 5 to about 5.7, about 5.1 to about 5.6. Variance of ±0.05 to ±0.4 pH units, ±0.1 to ±0.3 pH units or ±0.05 to ±0.25 pH units may be noted upon storage.

In some embodiments, a suitable buffer will have at least one pKa in a range of about 4.2 to about 5.5, about 4.3 to about 5.3. about 4.4 to about 5.2, about 4.5 to about 5.1 or about 4.6 to about 5.0. In some embodiments, particularly desirable buffer will have a pKa in the range of about 4.7 to 4.8. In this regard, it is noted that acetic acid is reported to have a pKa of 4.75 and citric acid has three titratable groups with pKa values of 3.13, 4.76 and 6.40, of which 4.76 is within a range considered desirable for preparing compositions according to the present invention. In some embodiments, other buffers may be used if they have the appropriate pKa and buffering capacity.

A buffer used in the formulations according to the present invention should possess sufficient buffering capacity to maintain the pH of the solution within predetermined limits during storage. Buffering capacity of an acid/base buffer system may be influenced by various factors, among them being the total concentration of the buffer, which is the total concentration of the protonated (acid) form of the buffer and each complementary base. In the case of a citrate buffer, the total concentration of the buffer should be at least about 30 mM, or at least about 45 mM or at least about 55 mM. In the case of acetate buffer, the total concentration of acetic acid and free acetate ion should be greater than about 70 mM, greater than about 80 mM or greater than about 90 mM. In some embodiments, the buffering capacity of the selected buffer should be sufficient to maintain pH in the range of about ±0.1, 0.2 or 0.3 pH units upon storage at 25° C. to about 45° C. for a period of 2, 4, 6, 8, 10, 12, 16, 20, 24 or more weeks.

### Antioxidants

In some embodiments, nasal compositions of metoclopramide may include one or more antioxidants suitable for administration to the nose or nasal cavity. In some embodiments, such antioxidants can include citric acid, citric acid monohydrate, sodium citrate dihydrate, butylated hydroxyanisole, or combinations of two or more thereof. In some particular embodiments, the antioxidant can include citric acid, citric acid monohydrate, and/or sodium citrate dihydrate. Thus, citric acid, citric acid monohydrate, and/or sodium citrate dihydrate can serve as both a buffering agent and as an antioxidant.

In various embodiments, the composition comprising metoclopramide, or a pharmaceutically-acceptable salt thereof, is substantially free of an additional antioxidant.

As used herein, “substantially free of any additional antioxidant” means that the solution contains no additional antioxidants other than those that are positively recited. In some embodiments, a solution may contain citrate, as defined herein, and be substantially free of any additional antioxidant.

As used herein, “as an antioxidant”, especially in reference to citric acid, means that the ingredient is added in order to impart its antioxidant value to the solution, and no conjugate salt of the ingredient (e.g., citric acid) or other pH adjuster (e.g., sodium hydroxide) is added to bring the solution to a particular pH. Thus, use of an ingredient “as an oxidant” is distinguished from use of the same ingredient as a buffer, where a particular pH or pH range is achieved by adding specific amounts of both acid and conjugate salt or base. This reflects the inventors' discovery that in some embodiments, addition of an acid ingredient, such as citric acid, to a solution results in stabilization of metoclopramide and protection of the solution against color change, and that in some embodiments it is not necessary to counter the acidity of said acid ingredient with a conjugate salt, or to form the conjugate salt in situ with addition of base, in order to achieve this stability and tendency not to change color.

### Particular Excipients

In some embodiments, nasal compositions of metoclopramide may include one or more particular excipients suitable for administration to the nose or nasal cavity. In some embodiments, such excipients can include citric acid, sodium citrate, benzalkonium chloride, sorbitol, sugar, edetate disodium dihydrate (EDTA), or combinations of two or more thereof. In some particular embodiments, the excipients can include citric acid and/or sodium citrate. In some embodiments, the excipients can include benzalkonium chloride or a combination of benzalkonium chloride and citric acid and/or sodium citrate. In some embodiments, the excipients can include a combination of benzalkonium chloride and sorbitol, optionally with the addition of one or both of citric acid and sodium citrate.

In some embodiments, the composition comprising metoclopramide, or a pharmaceutically-acceptable salt thereof, further comprises at least one member of the group consisting of a salt, edetate disodium dihydrate (EDTA), sorbitol, a sugar, and a flavoring agent.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. In some cases, the term “about” refers to ±10% of a stated number or value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “therapeutically effective amount” or “effective amount” refers to the amount of a compound or composition that, when administered, is sufficient to prevent development of, or alleviate to some extent, one or more of the symptoms of the disorder, disease, or condition being treated. The term “therapeutically effective amount” or “effective amount” also refers to the amount of a compound or composition that is sufficient to elicit the biological or medical response of a cell, tissue, system, animal, or human that is being sought by a researcher, veterinarian, medical doctor, or clinician.

The terms “nasal metoclopramide”, “intranasal composition comprising metoclopramide”, “nasal solution”, “metoclopramide nasal formulations”, “nasally administered metoclopramide”, “intranasally administering” or “intranasal” and the like relate to compounds, compositions, formulations for administration into the nose, e.g., via a nostril.

As used herein, “oral” means a dosage form taken by mouth, such as a tablet, powder, soft gel capsule, hard gel capsule, orally dissolving tablet or thin film, or liquid.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The section headings used herein are for organizational purposes only and are not to be construed as limiting the subject matter described.

### EXAMPLES

### Example 1

This example discloses a method for treating chronic gastroparesis in a patient in need thereof. The method comprises steps of: intranasally administering to the patient a composition comprising metoclopramide, or a pharmaceutically-acceptable salt thereof, for a first period of time; after the first period of time, not administering to the patient a composition comprising metoclopramide, or a pharmaceutically-acceptable salt thereof, for a second period of time; and after the second period of time, intranasally administering to the patient a composition comprising metoclopramide, or a pharmaceutically-acceptable salt thereof, for a third period of time.

The composition comprising metoclopramide may be any herein-disclose composition comprising metoclopramide.

In some cases, the method may comprise a fourth, a fifth, a sixth, or a seventh period of time which are similar to the first period of time or the second period of time, as described above.

The periods of time are as described elsewhere herein.

In this example, the patient does not experience tardive dyskinesia symptoms while the method is performed.

In this example, the patient does not experience adverse reaction symptoms while the method is performed. The adverse reaction is one or more of an extrapyramidal effect, a neuroleptic malignant syndrome, depression, hypertension, fluid retention, hyperprolactionemia, and the inability to drive or operate machinery.

In this example, the patient is a human, e.g., a female human. The patient, e.g., female patient, may have diabetic gastroparesis.

In this example, the patient in need thereof, has symptoms of nausea, bloating, emesis, delayed emesis, early satiety, vomiting, feeling full, loss of appetite, stomach fullness, stomach being visibly larger, and upper abdominal discomfort. In some cases, the symptoms are related to diabetic gastroparesis.

In this example, reducing the likelihood or probability is an at least 10% reduction in likelihood or probability, an least 20% reduction in likelihood or probability, an at least 30% reduction in likelihood or probability, an at least 40% reduction in likelihood or probability, an at least 50% reduction in likelihood or probability, an at least 60% reduction in likelihood or probability, an at least 70% reduction in likelihood or probability, an at least 80% reduction in likelihood or probability, or an at least 90% reduction in likelihood or probability. In some cases, reducing the likelihood or probability is an at least a one-fold reduction in likelihood or probability, at least a two-fold reduction in likelihood or probability, at least a three-fold reduction in likelihood or probability, at least a four-fold reduction in likelihood or probability, at least a five-fold reduction in likelihood or probability, at least a six-fold reduction in likelihood or probability, at least a seven-fold reduction in likelihood or probability, at least an eight-fold reduction in likelihood or probability, at least a nine-fold reduction in likelihood or probability, or at least a ten-fold reduction in likelihood or probability.

### Example 2

This example discloses a method for treating recurrent gastroparesis in a patient in need thereof. The method comprises steps of: intranasally administering to a patient a composition comprising metoclopramide, or a pharmaceutically-acceptable salt thereof. Here, the patient was previously intranasally administered a composition comprising metoclopramide, or a pharmaceutically-acceptable salt thereof, for a first period of time, and the patient was not intranasally administered a composition comprising metoclopramide, or a pharmaceutically-acceptable salt thereof, for a second period of time after the first period of time.

The composition comprising metoclopramide may be any herein-disclose composition comprising metoclopramide.

In some cases, the method may comprise a fourth, a fifth, a sixth, or a seventh period of time which are similar to the first period of time or the second period of time, as described above.

The periods of time are as described elsewhere herein.

In this example, the patient does not experience tardive dyskinesia symptoms while the method is performed.

In this example, the patient does not experience adverse reaction symptoms while the method is performed. The adverse reaction is one or more of an extrapyramidal effect, a neuroleptic malignant syndrome, depression, hypertension, fluid retention, hyperprolactionemia, and the inability to drive or operate machinery.

In this example, the patient is a human, e.g., a female human. The patient, e.g., female patient, may have diabetic gastroparesis.

In this example, the patient in need thereof, has symptoms of nausea, bloating, emesis, delayed emesis, early satiety, vomiting, feeling full, loss of appetite, stomach fullness, stomach being visibly larger, and upper abdominal discomfort. In some cases, the symptoms are related to diabetic gastroparesis.

In this example, reducing the likelihood or probability is an at least 10% reduction in likelihood or probability, an least 20% reduction in likelihood or probability, an at least 30% reduction in likelihood or probability, an at least 40% reduction in likelihood or probability, an at least 50% reduction in likelihood or probability, an at least 60% reduction in likelihood or probability, an at least 70% reduction in likelihood or probability, an at least 80% reduction in likelihood or probability, or an at least 90% reduction in likelihood or probability. In some cases, reducing the likelihood or probability is an at least a one-fold reduction in likelihood or probability, at least a two-fold reduction in likelihood or probability, at least a three-fold reduction in likelihood or probability, at least a four-fold reduction in likelihood or probability, at least a five-fold reduction in likelihood or probability, at least a six-fold reduction in likelihood or probability, at least a seven-fold reduction in likelihood or probability, at least an eight-fold reduction in likelihood or probability, at least a nine-fold reduction in likelihood or probability, or at least a ten-fold reduction in likelihood or probability.

### Example 3

This example discloses a method for reducing the likelihood or probability that a patient experiences tardive dyskinesia resulting from a metoclopramide therapy. The method comprises steps of: intranasally administering to the patient a composition comprising metoclopramide, or a pharmaceutically-acceptable salt thereof, for a first period of time; after the first period of time, not administering to the patient a composition comprising metoclopramide, or a pharmaceutically-acceptable salt thereof, for a second period of time; and after the second period of time, intranasally administering to the patient a composition comprising metoclopramide, or a pharmaceutically-acceptable salt thereof, for a third period of time.

The composition comprising metoclopramide may be any herein-disclose composition comprising metoclopramide.

In some cases, the method may comprise a fourth, a fifth, a sixth, or a seventh period of time which are similar to the first period of time or the second period of time, as described above.

The periods of time are as described elsewhere herein.

In this example, the patient does not experience tardive dyskinesia symptoms while the method is performed.

In this example, the patient does not experience adverse reaction symptoms while the method is performed. The adverse reaction is one or more of an extrapyramidal effect, a neuroleptic malignant syndrome, depression, hypertension, fluid retention, hyperprolactionemia, and the inability to drive or operate machinery.

In this example, the patient is a human, e.g., a female human. The patient, e.g., female patient, may have diabetic gastroparesis.

In this example, the patient in need thereof, has symptoms of nausea, bloating, emesis, delayed emesis, early satiety, vomiting, feeling full, loss of appetite, stomach fullness, stomach being visibly larger, and upper abdominal discomfort. In some cases, the symptoms are related to diabetic gastroparesis.

In this example, reducing the likelihood or probability is an at least 10% reduction in likelihood or probability, an least 20% reduction in likelihood or probability, an at least 30% reduction in likelihood or probability, an at least 40% reduction in likelihood or probability, an at least 50% reduction in likelihood or probability, an at least 60% reduction in likelihood or probability, an at least 70% reduction in likelihood or probability, an at least 80% reduction in likelihood or probability, or an at least 90% reduction in likelihood or probability. In some cases, reducing the likelihood or probability is an at least a one-fold reduction in likelihood or probability, at least a two-fold reduction in likelihood or probability, at least a three-fold reduction in likelihood or probability, at least a four-fold reduction in likelihood or probability, at least a five-fold reduction in likelihood or probability, at least a six-fold reduction in likelihood or probability, at least a seven-fold reduction in likelihood or probability, at least an eight-fold reduction in likelihood or probability, at least a nine-fold reduction in likelihood or probability, or at least a ten-fold reduction in likelihood or probability.

### Example 4

This example discloses a method for reducing the likelihood or probability that a patient experiences tardive dyskinesia resulting from a metoclopramide therapy. The method comprises steps of: intranasally administering to a patient a composition comprising metoclopramide, or a pharmaceutically-acceptable salt thereof. Here, the patient was previously intranasally administered a composition comprising metoclopramide, or a pharmaceutically-acceptable salt thereof, for a first period of time, and the patient was not intranasally administered a composition comprising metoclopramide, or a pharmaceutically-acceptable salt thereof, for a second period of time after the first period of time.

The composition comprising metoclopramide may be any herein-disclose composition comprising metoclopramide.

In some cases, the method may comprise a fourth, a fifth, a sixth, or a seventh period of time which are similar to the first period of time or the second period of time, as described above.

The periods of time are as described elsewhere herein.

In this example, the patient does not experience tardive dyskinesia symptoms while the method is performed.

In this example, the patient does not experience adverse reaction symptoms while the method is performed. The adverse reaction is one or more of an extrapyramidal effect, a neuroleptic malignant syndrome, depression, hypertension, fluid retention, hyperprolactionemia, and the inability to drive or operate machinery.

In this example, the patient is a human, e.g., a female human. The patient, e.g., female patient, may have diabetic gastroparesis.

In this example, the patient in need thereof, has symptoms of nausea, bloating, emesis, delayed emesis, early satiety, vomiting, feeling full, loss of appetite, stomach fullness, stomach being visibly larger, and upper abdominal discomfort. In some cases, the symptoms are related to diabetic gastroparesis.

In this example, reducing the likelihood or probability is an at least 10% reduction in likelihood or probability, an least 20% reduction in likelihood or probability, an at least 30% reduction in likelihood or probability, an at least 40% reduction in likelihood or probability, an at least 50% reduction in likelihood or probability, an at least 60% reduction in likelihood or probability, an at least 70% reduction in likelihood or probability, an at least 80% reduction in likelihood or probability, or an at least 90% reduction in likelihood or probability. In some cases, reducing the likelihood or probability is an at least a one-fold reduction in likelihood or probability, at least a two-fold reduction in likelihood or probability, at least a three-fold reduction in likelihood or probability, at least a four-fold reduction in likelihood or probability, at least a five-fold reduction in likelihood or probability, at least a six-fold reduction in likelihood or probability, at least a seven-fold reduction in likelihood or probability, at least an eight-fold reduction in likelihood or probability, at least a nine-fold reduction in likelihood or probability, or at least a ten-fold reduction in likelihood or probability.

### Example 5

This example discloses a method for reducing the likelihood or probability that a patient experiences an adverse reaction resulting from a metoclopramide therapy. The method comprises steps of: intranasally administering to the patient a composition comprising metoclopramide, or a pharmaceutically-acceptable salt thereof, for a first period of time; after the first period of time, not administering to the patient a composition comprising metoclopramide, or a pharmaceutically-acceptable salt thereof, for a second period of time; and after the second period of time, intranasally administering to the patient a composition comprising metoclopramide, or a pharmaceutically-acceptable salt thereof, for a third period of time. Here, the adverse reaction is one or more of an extrapyramidal effect, a neuroleptic malignant syndrome, depression, hypertension, fluid retention, hyperprolactionemia, and the inability to drive or operate machinery.

The composition comprising metoclopramide may be any herein-disclose composition comprising metoclopramide.

In some cases, the method may comprise a fourth, a fifth, a sixth, or a seventh period of time which are similar to the first period of time or the second period of time, as described above.

The periods of time are as described elsewhere herein.

In this example, the patient does not experience tardive dyskinesia symptoms while the method is performed.

In this example, the patient does not experience adverse reaction symptoms while the method is performed. The adverse reaction is one or more of an extrapyramidal effect, a neuroleptic malignant syndrome, depression, hypertension, fluid retention, hyperprolactionemia, and the inability to drive or operate machinery.

In this example, the patient is a human, e.g., a female human. The patient, e.g., female patient, may have diabetic gastroparesis.

In this example, the patient in need thereof, has symptoms of nausea, bloating, emesis, delayed emesis, early satiety, vomiting, feeling full, loss of appetite, stomach fullness, stomach being visibly larger, and upper abdominal discomfort. In some cases, the symptoms are related to diabetic gastroparesis.

In this example, reducing the likelihood or probability is an at least 10% reduction in likelihood or probability, an least 20% reduction in likelihood or probability, an at least 30% reduction in likelihood or probability, an at least 40% reduction in likelihood or probability, an at least 50% reduction in likelihood or probability, an at least 60% reduction in likelihood or probability, an at least 70% reduction in likelihood or probability, an at least 80% reduction in likelihood or probability, or an at least 90% reduction in likelihood or probability. In some cases, reducing the likelihood or probability is an at least a one-fold reduction in likelihood or probability, at least a two-fold reduction in likelihood or probability, at least a three-fold reduction in likelihood or probability, at least a four-fold reduction in likelihood or probability, at least a five-fold reduction in likelihood or probability, at least a six-fold reduction in likelihood or probability, at least a seven-fold reduction in likelihood or probability, at least an eight-fold reduction in likelihood or probability, at least a nine-fold reduction in likelihood or probability, or at least a ten-fold reduction in likelihood or probability.

### Example 6

This example discloses a method for reducing the likelihood or probability that a patient experiences an adverse reaction resulting from a metoclopramide therapy The method comprises steps of: intranasally administering to a patient a composition comprising metoclopramide, or a pharmaceutically-acceptable salt thereof. Here, the patient was previously intranasally administered a composition comprising metoclopramide, or a pharmaceutically-acceptable salt thereof, for a first period of time. Also, the patient is not intranasally administered a composition comprising metoclopramide, or a pharmaceutically-acceptable salt thereof, for a second period of time after the first period of time; and the adverse reaction is one or more of an extrapyramidal effect, a neuroleptic malignant syndrome, depression, hypertension, fluid retention, hyperprolactionemia, and the inability to drive or operate machinery.

The composition comprising metoclopramide may be any herein-disclose composition comprising metoclopramide.

In some cases, the method may comprise a fourth, a fifth, a sixth, or a seventh period of time which are similar to the first period of time or the second period of time, as described above.

The periods of time are as described elsewhere herein.

In this example, the patient does not experience tardive dyskinesia symptoms while the method is performed.

In this example, the patient does not experience adverse reaction symptoms while the method is performed. The adverse reaction is one or more of an extrapyramidal effect, a neuroleptic malignant syndrome, depression, hypertension, fluid retention, hyperprolactionemia, and the inability to drive or operate machinery.

In this example, the patient is a human, e.g., a female human. The patient, e.g., female patient, may have diabetic gastroparesis.

In this example, the patient in need thereof, has symptoms of nausea, bloating, emesis, delayed emesis, early satiety, vomiting, feeling full, loss of appetite, stomach fullness, stomach being visibly larger, and upper abdominal discomfort. In some cases, the symptoms are related to diabetic gastroparesis.

In this example, reducing the likelihood or probability is an at least 10% reduction in likelihood or probability, an least 20% reduction in likelihood or probability, an at least 30% reduction in likelihood or probability, an at least 40% reduction in likelihood or probability, an at least 50% reduction in likelihood or probability, an at least 60% reduction in likelihood or probability, an at least 70% reduction in likelihood or probability, an at least 80% reduction in likelihood or probability, or an at least 90% reduction in likelihood or probability. In some cases, reducing the likelihood or probability is an at least a one-fold reduction in likelihood or probability, at least a two-fold reduction in likelihood or probability, at least a three-fold reduction in likelihood or probability, at least a four-fold reduction in likelihood or probability, at least a five-fold reduction in likelihood or probability, at least a six-fold reduction in likelihood or probability, at least a seven-fold reduction in likelihood or probability, at least an eight-fold reduction in likelihood or probability, at least a nine-fold reduction in likelihood or probability, or at least a ten-fold reduction in likelihood or probability.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In particular, U.S. Pat. Nos. 6,770,262; 8,334,281; 11,020,361; 11,517,545; US 2013/0217775; US 2020/0276139; and US 2022/0151960 are incorporated herein by reference in their entirety. To the extent publications and patents or patent applications incorporated by reference contradict the disclosure contained in the specification, the specification is intended to supersede and/or take precedence over any such contradictory material.

What is claimed is:

1. A method for treating chronic gastroparesis in a patient in need thereof, the method comprising steps of:
intranasally administering to the patient a composition comprising metoclopramide, or a pharmaceutically-acceptable salt thereof, for a first period of time,
after the first period of time, not administering to the patient a composition comprising metoclopramide, or a pharmaceutically-acceptable salt thereof, for a second period of time; and
after the second period of time, intranasally administering to the patient a composition comprising metoclopramide, or a pharmaceutically-acceptable salt thereof, for a third period of time.

2. A method for treating recurrent gastroparesis in a patient in need thereof, the method comprising intranasally administering to a patient a composition comprising metoclopramide, or a pharmaceutically-acceptable salt thereof,
wherein the patient was previously intranasally administered a composition comprising metoclopramide, or a pharmaceutically-acceptable salt thereof, for a first period of time, and
wherein the patient was not intranasally administered a composition comprising metoclopramide, or a pharmaceutically-acceptable salt thereof, for a second period of time after the first period of time.

3. A method for reducing the likelihood or probability that a patient experiences tardive dyskinesia resulting from a metoclopramide therapy, the method comprising steps of:
intranasally administering to the patient a composition comprising metoclopramide, or a pharmaceutically-acceptable salt thereof, for a first period of time,
after the first period of time, not administering to the patient a composition comprising metoclopramide, or a pharmaceutically-acceptable salt thereof, for a second period of time; and
after the second period of time, intranasally administering to the patient a composition comprising metoclopramide, or a pharmaceutically-acceptable salt thereof, for a third period of time.

4. A method for reducing the likelihood or probability that a patient experiences tardive dyskinesia resulting from a metoclopramide therapy, the method comprising intranasally administering to a patient a composition comprising metoclopramide, or a pharmaceutically-acceptable salt thereof,
wherein the patient was previously intranasally administered a composition comprising metoclopramide, or a pharmaceutically-acceptable salt thereof, for a first period of time, and
wherein the patient was not intranasally administered a composition comprising metoclopramide, or a pharmaceutically-acceptable salt thereof, for a second period of time after the first period of time.

5. A method for reducing the likelihood or probability that a patient experiences an adverse reaction resulting from a metoclopramide therapy, the method comprising steps of:
intranasally administering to the patient a composition comprising metoclopramide, or a pharmaceutically-acceptable salt thereof, for a first period of time,
after the first period of time, not administering to the patient a composition comprising metoclopramide, or a pharmaceutically-acceptable salt thereof, for a second period of time; and
after the second period of time, intranasally administering to the patient a composition comprising metoclopramide, or a pharmaceutically-acceptable salt thereof, for a third period of time,
wherein the adverse reaction is one or more of an extrapyramidal effect, a neuroleptic malignant syndrome, depression, hypertension, fluid retention, hyperprolactionemia, and the inability to drive or operate machinery.

6. A method for reducing the likelihood or probability that a patient experiences an adverse reaction resulting from a metoclopramide therapy, the method comprising intranasally administering to a patient a composition comprising metoclopramide, or a pharmaceutically-acceptable salt thereof,
wherein the patient was previously intranasally administered a composition comprising metoclopramide, or a pharmaceutically-acceptable salt thereof, for a first period of time,
wherein the patient was not intranasally administered a composition comprising metoclopramide, or a pharmaceutically-acceptable salt thereof, for a second period of time after the first period of time; and
wherein the adverse reaction is one or more of an extrapyramidal effect, a neuroleptic malignant syndrome, depression, hypertension, fluid retention, hyperprolactionemia, and the inability to drive or operate machinery.

7. The method of any one of claims 1 to 6, wherein the sum of the first period of time and the third period of time is longer than 12 weeks.

8. The method of any one of claims 1 to 7, wherein the first period of time is longer than 12 weeks and/or the third period of time is longer than 12 weeks.

9. The method of any one of claims 1 to 7, wherein the first period of time of time is from about 2 weeks to about 8 weeks and/or the third period of time of time is from about 2 weeks to about 8 weeks.

10. The method of any one of claims 1 to 8, wherein the second period of time is at least 5 days, is at least 6 days, or is at least 7 days, is at least 8 days, is at least 9 days, is at least 10 days, is at least 12 days, is at least 13 days, is at least 2 weeks, is at least 3 weeks, is at least 4 weeks, is at least two months, or is at least three months.

11. The method of any one of claims 1 to 10, wherein the method further comprising a step of:
after the third period of time, not administering to the patient a composition comprising metoclopramide, or a pharmaceutically-acceptable salt thereof, for a fourth period of time.

12. The method of claim 11, wherein the method further comprising a step of:
after the fourth period of time, intranasally administering to the patient a composition comprising metoclopramide, or a pharmaceutically-acceptable salt thereof, for a fifth period of time.

13. The method of claim 11 or claim 12, wherein the fourth period of time is at least 5 days, is at least 6 days, or is at least 7 days, is at least 8 days, is at least 9 days, is at least 10 days, is at least 12 days, is at least 13 days, is at least 2 weeks, is at least 3 weeks, is at least 4 weeks, is at least two months, or is at least three months.

14. The method of claim 12 or claim 13, wherein the fifth period of time is longer than 12 weeks or wherein the fifth period of time is from about 2 weeks to about 8 weeks.

15. The method of any one of claims 11 to 14, wherein the method further comprising a step of:
after the fifth period of time, not administering to the patient a composition comprising metoclopramide, or a pharmaceutically-acceptable salt thereof, for a sixth period of time.

16. The method of claim 15, wherein the method further comprising a step of:
after the sixth period of time, intranasally administering to the patient a composition comprising metoclopramide, or a pharmaceutically-acceptable salt thereof, for a seventh period of time.

17. The method of claim 15 or claim 16, wherein the sixth period of time is at least 5 days, is at least 6 days, or is at least 7 days, is at least 8 days, is at least 9 days, is at least 10 days, is at least 12 days, is at least 13 days, is at least 2 weeks, is at least 3 weeks, is at least 4 weeks, is at least two months, or is at least three months.

18. The method of claim 16 or claim 17, wherein the seventh period of time is longer than 12 weeks or the seventh period of time is from about 2 weeks to about 8 weeks.

19. The method of any one of claims 1 to 18, wherein the method is performed for greater than 12 weeks.

20. The method claim 19, wherein the method is performed for six months or longer.

21. The method claim 20, wherein the method is performed for one year or longer.

22. The method claim 21, wherein the method is performed for two years or longer.

23. The method claim 22, wherein the method is performed for three years or longer.

24. The method claim 23, wherein the method is performed for four years or longer.

25. The method claim 24, wherein the method is performed for five years or longer.

26. The method of any one of claims 2 to 25, wherein reducing the likelihood or probability is an at least 10% reduction in likelihood or probability relative to a method lacking, at least, a second period of time.

27. The method of any one of claims 11 to 26, wherein reducing the likelihood or probability is an at least 10% reduction in likelihood or probability relative to a method lacking, at least, a fourth period of time.

28. The method of any one of claims 15 to 27, wherein reducing the likelihood or probability is an at least 10% reduction in likelihood or probability relative to a method lacking, at least, a sixth period of time.

29. The method of any one of claims 26 to 28, wherein reducing the likelihood or probability is an at least 20% reduction in likelihood or probability, an at least 30% reduction in likelihood or probability, an at least 40% reduction in likelihood or probability, an at least 50% reduction in likelihood or probability, an at least 60% reduction in likelihood or probability, an at least 70% reduction in likelihood or probability, an at least 80% reduction in likelihood or probability, or an at least 90% reduction in likelihood or probability.

30. The method of any one of claims 26 to 29, wherein reducing the likelihood or probability is an at least a one-fold reduction in likelihood or probability, an at least a two-fold reduction in likelihood or probability, an at least a three-fold reduction in likelihood or probability, an at least a four-fold reduction in likelihood or probability, an at least a five-fold reduction in likelihood or probability, an at least a six-fold reduction in likelihood or probability, an at least a seven-fold reduction in likelihood or probability, an at least an eight-fold reduction in likelihood or probability, an at least a nine-fold reduction in likelihood or probability, or an at least a ten-fold reduction in likelihood or probability.

31. The method of any one of claims 1 to 30, wherein the patient does not experience tardive dyskinesia symptoms while the method is performed.

32. The method of any one of claims 1 to 31, wherein the patient does not experience adverse reaction symptoms while the method is performed,
wherein the adverse reaction is one or more of an extrapyramidal effect, a neuroleptic malignant syndrome, depression, hypertension, fluid retention, hyperprolactionemia, and the inability to drive or operate machinery.

33. The method of any one of claims 1 to 32, wherein the composition comprising metoclopramide, or a pharmaceutically-acceptable salt thereof, further comprises benzalkonium chloride.

34. The method of claim 33, wherein the composition has a concentration of benzalkonium chloride from about 0.005% (w/v) to about 0.05% (w/v).

35. The method of claim 34, wherein the benzalkonium chloride is at a concentration of from about 0.02% to about 0.04% (w/v).

36. The method of claim 35, wherein the benzalkonium chloride is at a concentration of from about 0.02% to about 0.03% (w/v).

37. The method of claim 36, wherein the benzalkonium chloride is at a concentration of at least about 0.025% (w/v).

38. The method of claim 37, wherein the benzalkonium chloride is at a concentration of about 0.025% (w/v).

39. The method of any one of claims 1 to 38, wherein the composition comprising metoclopramide, or a pharmaceutically-acceptable salt thereof, further comprises benzyl alcohol.

40. The method of claim 39, wherein the benzyl alcohol is at a concentration from about 0.01% (w/v) to about 1% (w/v).

41. The method of claim 39 or claim 40, wherein the benzyl alcohol is at a concentration of about 0.75% (w/v).

42. The method of any one of claims 1 to 41, wherein the composition comprising metoclopramide, or a pharmaceutically-acceptable salt thereof, further comprises a buffer.

43. The method of claim 42, wherein the buffer is selected from the group consisting of citric acid/phosphate, acetate, barbital, borate, Britton-Robinson, cacodylate, citrate, collidine, formate, maleate, McIlvaine, phosphate, Prideaux-Ward, succinate, citrate-phosphate-borate (Teorell-Stanhagen), veronal acetate, IVIES (2-(N-morpholino)ethanesulfonic acid), BIS-TRIS (bis(2-hydroxyethyl)iminotris(hydroxymethyl)methane), ADA (N-(2-acetamido)-2-iminodiacetic acid), ACES (N-(carbamoylmethyl)-2-aminoethanesulfonaic acid), PIPES (piperazine-N,N′-bis(2-ethanesulfonic acid)), MOPSO (3-(N-morpholino)-2-hydroxypropanesulfonic acid), BIS-TRIS PROPANE (1,3-bis(tris(hydroxymethyl)methylamino)propane), BES (N,N-bis(2-hydroxyethyl)-2-aminoethanesulfonaic acid), MOPS (3-(N-morpholino)propanesulfonic acid), TES (N-tris(hydroxymethyl)methyl-2-aminoethanesulfonic acid), HEPES (N-(2-hydroxyethyl)piperazine-N′-(2-ethanesulfonic acid), DIPSO (3-(N,N-bis(2-hydroxyethyl)amino)-2-hydroxypropanesulfonic acid), MOBS (4-(N-morpholino)butanesulfonic acid), TAPSO (3-(N-tris(hydroxymethyl)methylamino)-2-hydroxy-propanesulfonic acid), tris(hydroxymethylaminomethane, HEPPSO (N-(2-hydroxyethyl)piperazine-N′-(2-hydroxypropanesulfonic acid), POPSO (piperazine-N,N′-bis(2-hydroxypropanesulfonic acid)), TEA (triethanolamine), EPPS (N-(2-hydroxyethyl)piperazine-N′-(3-propane-sulfonic acid), TWINE (N-tris(hydroxymethyl)methylglycine), GLY-GLY (glycylglycine), BICINE (N,N-bis(2-hydroxyethyl)glycine), HEPBS (N-(2-hydroxyethyl)piperazine-N′-(4-butanesulfonic acid)), TAPS (N-tris(hydroxy-methypmethyl-3-aminopropanesulfonic acid), and AMPD (2-amino-2-methyl-1,3-propanediol) buffer.

44. The method of claim 42 or claim 43, wherein the buffer comprises a citrate buffer.

45. The method of claim 44, wherein the citrate buffer comprises a combination of citric acid monohydrate and sodium citrate dihydrate.

46. The method of claim 45, wherein the citric acid monohydrate is in an amount of from about 0.2% to about 0.5% w/v, from about 0.25% to about 0.4% w/v, or from about 0.3% to about 0.35% w/v and the sodium citrate dihydrate is in an amount from about 1.0 to about 1.8% w/v, from about 1.2 to about 1.6% w/v, or from about 1.3 to about 1.5% w/v.

47. The method of claim any one of claim 45 or claim 46, wherein a combined amount of citric acid monohydrate and sodium citrate dihydrate in the composition is less than about 2.3% w/v.

48. The method of any one of claims 45 to 47, wherein the citric acid monohydrate is in an amount of about 0.1% and the sodium citrate dihydrate is in an amount of about 0.44%.

49. The method of any one of claims 45 to 48, wherein the composition provides a citrate concentration of at least about 10 millimolar.

50. The method of any one of claims 42 to 49, wherein the buffer comprises sodium acetate.

51. The method of any one of claims 1 to 50, wherein the composition comprising metoclopramide, or a pharmaceutically-acceptable salt thereof, has a pH of above about 4.5.

52. The method of claim 51, wherein the composition has a pH of above about 4.6.

53. The method of claim 52, wherein the composition has a pH of above about 5.0.

54. The method of any one of claims 1 to 53, wherein the composition comprising metoclopramide, or a pharmaceutically-acceptable salt thereof, further comprises at least one member of the group consisting of a salt, edetate disodium dihydrate (EDTA), sorbitol, a sugar, and a flavoring agent.

55. The method of any one of claims 1 to 54, wherein the composition comprising metoclopramide, or a pharmaceutically-acceptable salt thereof, is substantially free of an additional antioxidant.

56. The method of any one of claims 1 to 55, wherein the composition has a concentration of metoclopramide, or a pharmaceutically-acceptable salt thereof, of from about 20.0% (w/v) to about 30.0% (w/v).

57. The method of claim 56, wherein the composition comprises 5 mg to 25 mg of metoclopramide, or a pharmaceutically-acceptable salt thereof, per aliquot.

58. The method of claim 56 or claim 57, wherein the metoclopramide composition comprises about 5 mg, about 5.5 mg, about 6 mg, about 6.5 mg, about 7 mg, about 7.5 mg, about 8 mg, about 8.5 mg, about 9 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, of metoclopramide, or a pharmaceutically-acceptable salt thereof, per aliquot.

59. The method of any one of claims 56 to 58, wherein a dose of 20 mg to 100 mg of metoclopramide, or a pharmaceutically-acceptable salt thereof, is administered per day.

60. The method of any one of claims 56 to 59, wherein a dose of 30 mg to 80 mg of metoclopramide, or a pharmaceutically-acceptable salt thereof, is administered per day.

61. The method of any one of claims 56 to 60, wherein a dose of 30 mg to 60 mg of metoclopramide, or a pharmaceutically-acceptable salt thereof, is administered per day.

62. The method of any one of claims 56 to 61, wherein a dose of 30 mg to 45 mg of metoclopramide, or a pharmaceutically-acceptable salt thereof, is administered per day.

63. The method of any one of claims 1 to 62, wherein the composition comprising metoclopramide, or a pharmaceutically-acceptable salt thereof, is administered as at least two aliquots per day.

64. The method of claim 63, wherein the composition is administered as three aliquots per day.

65. The method of claim 63, wherein the composition is administered as four aliquots per day.

66. The method of any one of claims 1 to 62, wherein the composition comprising metoclopramide, or a pharmaceutically-acceptable salt thereof, is administered as an intranasal spray.

67. The method of any one of claims 1 to 66, wherein an aliquot of the composition comprising metoclopramide, or a pharmaceutically-acceptable salt thereof, has a volume of from about 25 μL to about 140 μL.

68. The method of claim 67, wherein the aliquot of the composition has a volume of about 50 μL.

69. The method of claim 67, wherein the aliquot of the composition has a volume of about 70 μL.

70. The method of any one of claims 1 to 69, wherein the composition comprising metoclopramide, or a pharmaceutically-acceptable salt thereof, is administered as one spray in one nostril, from about 15 minutes to about 1 hour before a meal.

71. The method of claim 70, wherein the composition is administered as one spray in one nostril, from about 20 minutes to about 45 minutes before a meal.

72. The method of claim 71, wherein the composition is administered as one 1 spray in one nostril, about 30 minutes before a meal.

73. The method of any one of claims 1 to 72, wherein the composition comprising metoclopramide, or a pharmaceutically-acceptable salt thereof, has an osmolality of from about 500 mOsm/kg to about 1400 mOsm/kg.

74. The method of any one of claims 1 to 73, wherein the composition comprising metoclopramide, or a pharmaceutically-acceptable salt thereof, comprises benzalkonium chloride, citric acid monohydrate, edetate disodium dihydrate (EDTA), purified water, sodium citrate dihydrate, and sorbitol.

75. The method of claim 74, wherein each 70 μL aliquot of the composition comprises 15 mg of metoclopramide, or a pharmaceutically-acceptable salt thereof; each 70 μL aliquot of the composition comprises 7.5 mg of metoclopramide, or a pharmaceutically-acceptable salt thereof; or each 35 μL aliquot of the composition comprises 7.5 mg of metoclopramide.

76. The method of claim 74 or claim 75, wherein the composition has a pH of about 5.5.

77. The method of any one of claims 74 to 76, wherein the composition has a citrate concentration ([citrate]=[citric acid]+[dihydrogen citrate ion]+[hydrogen citrate ion]+[citrate ion]) of at least about 10 millimolar.

78. The method of any one of claims 1 to 73, wherein the composition comprising metoclopramide, or a pharmaceutically-acceptable salt thereof, comprises benzyl alcohol, citric acid monohydrate, edetate disodium dihydrate (EDTA), purified water, sodium citrate dihydrate, and sorbitol.

79. The method of claim 78, wherein the composition comprises less than about 1% w/v benzyl alcohol.

80. The method of any one of claims 1 to 73, wherein the composition comprising metoclopramide, or a pharmaceutically-acceptable salt thereof, comprises benzyl alcohol, an acetate buffer, edetate disodium dihydrate (EDTA), purified water, and sorbitol.

81. The method of any one of claims 1 to 80, wherein the composition comprising metoclopramide, or a pharmaceutically-acceptable salt thereof, exhibits less than about 2% average change in percent optical density (O.D.) per week per 200 mg/mL of metoclopramide when stored at a temperature of 40° C. and 75% relative humidity.

82. The method of claim 81, wherein the average change of percent optical density (O.D.) is less than about 1.8% O.D. per week.

83. The method of any one of claims 1 to 82, wherein the composition comprising metoclopramide, or a pharmaceutically-acceptable salt thereof, is a nasal solution that remains clear to pale yellow when compared to standard E, 32 USP <631> on storage at a temperature of about 40° C. for at least about 8 weeks.

84. The method of any one of claims 1 to 83, wherein the patient in need thereof, has symptoms of nausea, bloating, emesis, delayed emesis, early satiety, vomiting, feeling full, loss of appetite, stomach fullness, stomach being visibly larger, and upper abdominal discomfort.

85. The method of any one of claims 1 to 84, wherein the patient in need thereof, is a human.

86. The method of claim 85, wherein the human is a female.

87. The method of claim 85 or claim 86, wherein the human is an adult.

88. The method of any one of claims 1 to 87, wherein the patient has diabetic gastroparesis.
